(19) United States
(12) Patent Application Publication
(10) Pub. No.: US 2006/0286061 A1
(43) Pub. Date: Dec. 21, 2006

(54) Amide esters as hydrocarbon gellants

(76) Inventors: Kevin A. O'Lenick, Dacula, GA (US); Anthony J. O'Lenick JR., Dacula, GA (US)

Correspondence Address: ANTHONY J O'LENICK JR, 2170 LUKE EDWARDS ROAD, DACULA, GA, 30019, US

(21) Appl. No.: 11/450,847

(22) Filed: Jun. 12, 2006

Related U.S. Application Data
(60) Provisional application No. 60/691,814, filed on Jun. 20, 2005.

(51) Int. Cl. A61K 8/42; C07C 237/02

(52) U.S. Cl. 424/70.31; 554/63

(57) ABSTRACT

The invention relates to gelling agents, and in particular to gellants for low polarity liquids such as hydrocarbons, and the use thereof in commercial products, e.g., personal care products.

### RELATED APPLICATION

This application claims priority to and benefit of U.S. Provisional Application Nos. 60/691,814 filed Jun. 20, 2005, the disclosures of each of which are incorporated herein for all purposes.

### FIELD OF THE INVENTION

The invention relates to gelling agents, and in particular to gellants for low polarity liquids such as hydrocarbons, and the use thereof in commercial products, e.g., personal care products. The compositions of the present invention make use of simple amid diesters and do not require the complicated resin polymers taught previously.

### BACKGROUND OF THE INVENTION

Personal care products generally contain one or more active ingredients within a carrier formulation. While the active ingredient(s) determine the ultimate performance properties of the product, the carrier formulation is equally critical to the commercial success of the product. The rheology of the carrier (also referred to as the “base”) largely determines the flow properties of the product, and the flow properties largely determine the manner in which the consumer will apply or use the product.

For example, aluminum chlorohydrate and aluminum-zirconium tetrachlorohydrexgly are metal salts that are commonly used as active ingredients in deodorant and antiperspirant products. Consumers have shown a preference for applying deodorant from a stick form. Thus, the carrier in a stick-form deodorant must be a relatively hard substance, and waxy fatty alcohol such as stearyl alcohol has been used as the carrier in these products. As another example, the active ingredient in a lipstick is the colorant. A lipstick should not be as hard as a stick deodorant, but of course must maintain its shape when undisturbed at room temperature. A blend of wax and oil is known to provide a consistency that is well-suited as a carrier for a lipstick. As a final example, shampoo desirably has a viscosity greater than water, and when the active ingredient(s) in a shampoo does not have a sufficiently high viscosity, a somewhat viscous carrier material is desirably included in the shampoo formulation.

From the above examples, it is seen that formulators of personal care products depend upon the availability of materials having various rheological properties, in order to formulate a successful personal care product. Materials which have a gel-like character, in that they maintain their shape when undisturbed but flow upon being rubbed, are often desired for personal care products.

Transparent (i.e., clear) carriers are needed by formulators who develop a personal care product wherein colorant is an active ingredient, because a transparent carrier (as opposed to an opaque carrier) will minimally, if at all, interfere with the appearance of the colorant. However, in recent years consumers have demonstrated an increasing preference for transparent personal care products such as deodorants and shampoos. There is thus an increasing demand for transparent materials which can provide the Theological properties needed for various personal care products, and particularly which can impart gel-like character to a formulation.

Polyamide resin prepared from polymerized fatty acid and diamine is reported to function as a gellant in formulations developed for personal care products. For example, U.S. Pat. No. 3,148,125 is directed to a clear lipstick composition formed from polyamide resin compounded with a lower aliphatic alcohol and a so-called “polyamide solvent.” Likewise, U.S. Pat. No. 5,500,209 is directed to forming a gel or stick deodorant, where the composition contains polyamide gelling agent and a solvent system including monohydric or polyhydric alcohols. Thus, the prior art recognizes to blend certain polyamides with alcohols, to thereby form a gel.

Certain modified polyamide resins, e.g., polyamides which are only partly amidated but contain esterified carboxyl groups, have been reported to impart high gel strength and pronounced thixotropic properties to coating compositions that contain alkyd resins or drying oils. See U.S. Pat. No. 3,141,767 to Goetze et al. However, the modified polyamide resins of Goetze et al. are not disclosed as being useful gellants in personal care products, nor useful gellants when a low polarity fluid is used as the vehicle.

Low polarity fluids are desirably included in a personal care formulation because they are often transparent, relatively inexpensive, and non-toxic. Low polarity fluids are also available in a wide variety of viscosities and grades. However, low polarity fluids often do not have the rheological properties that are desired in a carrier, e.g., they do not naturally exhibit gel-like character. There is a need in the art for materials that can be combined with low polarity solvent, such as a hydrocarbon or fatty acid ester, to afford a transparent material which has gel-like character. The gel-like character is preferably of a smooth, silky feeling when the gel is rubbed against the skin. The present invention provides this and related advantages as described herein.

U.S. Pat. No. 6,875,245 to Pavlin, incorporated herein by reference in its entirety, issued Apr. 5, 2005 discloses “a resin composition is prepared by reacting components comprising dibasic acid, diamine, polyol and monoalcohol, wherein (a) at least 50 equivalent percent of the dibasic acid comprises polymerized fatty acid; (b) at least 50 equivalent percent of the diamine comprises ethylene diamine; (c) 10-60 equivalent percent of the total of the hydroxyl and amine equivalents provided by diamine, polyol and monoalcohol are provided by monoalcohol; and (d) no more than 50 equivalent percent of the total of the hydroxyl and amine equivalents provided by diamine, polyol and monoalcohol are provided by polyol. This resin composition may be formulated into, for example, personal care products, fragrance releasing products and candles. “These so called ester-terminated poly(ester-amides) in are used in personal care products. The Pavlin reference indicates the state of thought as to what constitutes a proper gellant for hydrocarbon materials. The compounds are complex polyamides capped with either esters of amides. These compounds are the result of complex reactions and the effectiveness of such materials is dependant upon the molecular weight, the molecular distribution and process used to make them.

This is in stark contrast to the present invention. We have surprisingly and unrepentantly found that simple properly branched amid esters made by the reaction of monoethanolamine and fatty acids provides products with outstanding thickening properties that provide clear gels in hydrocarbon at as low as 1-5% by weight.

Without wanting to be bound by one specific theory, we believe that the functioning of these gellants is based upon the proper selection of a product that is an amphorous solid having a high melting point and solubility in hydrocarbon. The fact that the gellant is not crystalline provides the needed gelling properties. Products which tend to be very crystalline fail to provide clear gels. The branching through nitrogen provides such a non-crystalline product.

There has been a long felt unsatisfied need in the personal care market to provide simple materials that would effectively provide a clear gel in hydrocarbon systems. The polyamide resins of Pavlin are amphorous high melting materials, but are not unlike the compositions of the present invention are complicated to make and cap and can vary in effectiveness.

Pavlin states “It is important to control the stoichiometry of the reactants in order to prepare ester-terminated poly(ester-amides) according to the invention. In the following discussion regarding reactant stoichiometry, the terms “equivalent(s)” and “equivalent percent” will be used, and are intended to have their standard meanings as employed in the art. However, for additional clarity, it is noted that equivalents refer to the number of reactive groups present in a molar quantity of a molecule, such that a mole of a dibasic acid (e.g., sebacic acid) has two equivalents of carboxylic acid, while a mole of monoalcohol has one equivalent of hydroxyl. Furthermore, it is emphasized that the dibasic acid has only two reactive groups (both carboxylic acids), the monoalcohol has only one reactive group (a hydroxyl group), the diamine has only two reactive groups (preferably both primary amines), and the polyol has at least two reactive groups (i.e., at least two reactive hydroxyl groups) and these are preferably, although not necessarily, the only reactive materials present in the reaction mixture.”

The compounds of the present invention are not resins, but simply fully reacted branched amid esters. The Pavlin reference teaches away from the effectiveness of such simple compounds, relying upon complex resins.

### SUMMARY OF THE INVENTION

One aspect, the present invention provides a novel branched amid esters. This branching destroys the crystallinity of the resulting material providing a gellant.

Another aspect, the present invention provides a gelled composition comprising

(i) an amid ester

(ii) a hydrocarbon

Still another aspect, the present invention provides a process for the gellation of composition comprising

(i) an amid ester

(ii) hydrocarbon

which comprises addition of between 1% and 35% by weight of a branched amid ester.

### DETAILED DESCRIPTION OF THE INVENTION

The present invention provides an amid ester conforming to the following structure;
R—C(O)—N(H)—CH₂CH₂—OC(O)—R
wherein;
R is selected from the group consisting of
(a) alkyl having 16 to 22 carbon atoms;
(b) iso acids;
(b) Guerbet acids 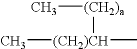
a is an integer ranging from 9 to 17
b is an integer ranging from 7 to 17 with the proviso that b=a+2.

Another aspect, the present invention provides a gelled composition comprising
(i) an amid ester conforming to the following structure;
   R—C(O)—N(H)—CH₂CH₂—OC(O)—R
   wherein;
R is selected from the group consisting of
(a) alkyl having 16 to 22 carbon atoms;
(b) iso acids;
(b) Guerbet acids 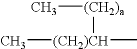
a is an integer ranging from 9 to 17
b is an integer ranging from 7 to 17 with the proviso that b=a+2.
(ii) a hydrocarbon selected from the group consisting of isohexadecane, isoeicosane, polyisobutene, isooctane, isododecane, hexane, decane, dodecane.

The present invention provides a process for the gellation of composition comprising
R—C(O)—N(H)—CH₂CH₂—OC(O)—R
wherein;
R is selected from the group consisting of
(a) alkyl having 16 to 22 carbon atoms;
(b) iso acids;
(b) Guerbet acids 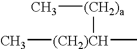
a is an integer ranging from 9 to 17
b is an integer ranging from 7 to 17 with the proviso that b=a+2.
(ii) a hydrocarbon selected from the group consisting of isohexadecane, isoeicosane, polyisobutene, isooctane, isododecane, hexane, decane, dodecane which comprises addition of between 1 and 35% by weight of amid ester.

Accordingly, another aspect of the invention is a gel formed between ingredients comprising the amid ester as described above and a non-aqueous liquid, preferably a low-polarity liquid. A preferred low polarity liquid is a hydrocarbon, with preferred hydrocarbons being solvents and oils. Solvents and oils may be distinguished in that defatting occurs when solvents are rubbed on human skin, leading to drying and irritation. However, defatting does not occur when oils are rubbed on human skin. Oils are more preferred than solvents in most personal-care formulations, and thus are preferred in forming the gels of the present invention. Preferably, the hydrocarbon has a relatively high number of carbon atoms, e.g., 10 to 30 carbon atoms, and thus is not a volatile hydrocarbon.

A preferred oil is mineral oil, also sometimes referred to as medicinal oil. Mineral oil is a highly refined, colorless, tasteless, and odorless petroleum oil (i.e., derived by processing petroleum/crude oil) used medicinally as an internal lubricant and for the manufacture of salves and ointments. Such mineral oils are highly refined in having substantially all volatile hydrocarbons removed there from, and in being hydrogenated (also called hydrotreated) in order to remove substantially all unsaturation, e.g., aromatic groups have been reduced to the fully saturated analog. A preferred mineral oil to prepare a gel of the invention is so-called “white” mineral oil, which is water-white (i.e., colorless and transparent) and is generally recognized as safe for contact with human skin. Mineral oil may also be characterized in terms of its viscosity, where light mineral oil is relatively less viscous than heavy mineral oil, and these terms are defined more specifically in the U.S. Pharmacopoeia, 22.sup.nd revision, p. 899 (1990). Any mineral oil may be used in the invention to form a gel.

Other hydrocarbons that may be used in the invention include relatively lower molecular weight hydrocarbons including linear saturated hydrocarbons such a tetradecane, hexadecane, octadecane, etc. Cyclic hydrocarbons such as decahydronaphthalene (DECALIN), fuel grade hydrocarbons, branched chain hydrocarbons such as PERMETHYL from Permethyl Corporation and ISOPAR from Exxon Corp., and hydrocarbon mixtures such as product PD-23 from Witco (Greenwich, Conn.) may also be used in preparing gels of the invention. Such hydrocarbons, particularly saturated hydrocarbon oils, are a preferred liquid for preparing a gel of the invention because such hydrocarbons are often less irritating to the skin than liquids containing aromatic, ketone and other functional groups.

Another class of suitable low polarity liquids are esters, and particularly esters of fatty acids. Such esters may be monofunctional esters (i.e., have a single ester moiety) or may be polyfunctional (i.e., have more than one ester group). Suitable esters include, but are not limited to, the reaction products of C₁₋₂₄ monoalcohols with C₁₋₂₂ monocarboxylic acids, where the carbon atoms may be arranged in a linear, branched and/or cyclic fashion, and unsaturation may optionally be present between carbon atoms. Preferably, the ester has at least about 18 carbon atoms. Examples include, but are not limited to, fatty acid esters such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate and tetratriacontanyl stearate; salicylates, e.g., C₁₋₁₀ salicylates such as octyl salicylate, and benzoate esters including C₁₂₋₁₅ alkyl benzoate, isostearyl benzoate and benzyl benzoate.

Suitable esters are those commonly employed in the cosmetics industry for the formulation of lipstick and make-up, e.g., the fatty acid esters mentioned above, and are often denoted as “cosmetic esters.” Other cosmetic esters include glycerol and propylene glycol esters of fatty acids, including the so-called polyglycerol fatty acid esters and triglycerides. Exemplary cosmetic esters include, without limitation, propylene glycol monolaurate, polyethylene glycol (400) monolaurate, castor oil, triglyceryl diisostearate and lauryl lactate. Thus, the liquid may have more than one of ester, hydroxyl and ether functionality. For example, C₁₀₋₁₅ alkyl lactate may be used in a gel of the invention. In addition, esterified polyols such as the polymers and/or copolymers of ethylene oxide, propylene oxide and butylene oxide reacted with C₁₋₂₂ monocarboxylic acids are useful. The carbon atoms of the C₁₋₂₂ monocarboxylic acids may be arranged in a linear, branched and/or cyclic fashion, and unsaturation may be present between the carbon atoms. Preferred esters are the reaction product of an alcohol and a fatty acid, where the alcohol is selected from C₁₋₁₀ monohydric alcohol, C₂₋₁₀ dihydric alcohol and C₃₋₁₀ trihydric alcohol, and the fatty acid is selected from a C₈₋₂₄ fatty acid.

The gels of the invention are preferably self-supporting, in that they retain their shape at room temperature and in the absence of shear. Also, the inventive gels are preferably clear or translucent. The terms clear, transparent and clarity are intended to have their ordinary dictionary definitions; thus, a clear gel allows ready viewing of objects behind it. By contrast, a translucent gel, although allowing light to pass through, causes the light to be so scattered that it will be impossible to see clearly objects behind the translucent stick. As used herein, a gel is transparent or clear if the maximum transmittance of light of any wavelength in the range 400 to 800 nm through a sample 1 cm thick is at least 35%, preferably at least 50% (see, e.g., European Patent Publication No. 291,334 A4). The gel is translucent if the maximum transmittance of such light through the sample is between 2% and less than 35%. The transmittance can be measured by placing a sample of the aforementioned thickness into a light beam of a spectrophotometer whose working range includes the visible spectrum, such as a Bausch & Lomb Spectronic 88 Spectrophotometer.

The gels of the invention preferably do not display syneresis. As defined in the McGraw-Hill Dictionary of Scientific and Technical Terms (3.sup.rd Edition), syneresis is the spontaneous separation of a liquid from a gel or colloidal suspension due to contraction of the gel. Typically, syneresis is observed as the separation of liquid from a gel, and is sometimes referred to as “bleeding,” in that wetness is seen along the surfaces of a gel that displays syneresis. From a commercial point of view, syneresis is typically an undesirable property, and the gels of the present invention desirably, and surprisingly do not exhibit syneresis.

To prepare a gel of the invention, the amid ester is combined with a liquid. The two ingredients are taken to elevated temperature, e.g., up to about 80-150° C., until the amid ester is completely dissolves in the liquid. A lower temperature may be used if a solution can be prepared at the lower temperature. Upon cooling, the mixture forms the gel of the invention. Preferably, the liquid is a low-polarity liquid as described above, and more preferably the liquid is a hydrocarbon. The liquid may contain more than one component, e.g., hydrocarbon as well as ester-containing material. In any event, the amid ester is combined with the liquid such that the weight percent of the amid ester in the branched diamid+solvent mixture is about 5-50%, and preferably is about 10-45%. Such gels may be transparent, translucent or opaque, depending on the precise identities of the branched diamid and liquid, as well as the concentration of the amid ester in the mixture.

A commercially desirable aspect of the invention is that the gel may be (although need not be) essentially transparent. Thus, the gels are desirably combined with colorants, as well as other ingredients, to form lipstick and other cosmetic products. The advantage of a clear gel in these applications is that the gel imparts little if any undesirable color to the lipstick or cosmetic. The gels may be combined with aluminum zirconium salts, as well as other ingredients, to form colorless underarm deodorant/antiperspirant, which is currently quite popular. The gels of the invention are also useful in other personal care products, e.g., cosmetics such as eye make-up, lipstick, foundation make-up, costume make-up, as well as baby oil, make-up removers, bath oil, skin moisturizers, sun care products, lip balm, waterless hand cleaner, medicated ointments, ethnic hair care products, perfume, cologne, and suppositories. In addition, the gels may be used in household products such as automobile wax/polish, candles, furniture polish, metal cleaners/polishes, household cleaners, paint strippers and insecticide carriers.

The gels may also be used in industrial products such as fuels (sterno, lighters), toilet bowl rings, lubricants/greases, wire rope lubricant, joint and cable fillers, soldering flux, buffing compounds, crayons and markers, modeling clay, rust preventatives, printing inks, protective/removable coatings, and jet inks. For example, a hydrocarbon oil gelled with an amid ester of the invention may be used as a heat source in, e.g., a cooking apparatus used in camping and hiking. Such a composition will not flow if tilted, and thus may be safer and neater than similar products made from flowing materials.

Formulations to prepare such materials are well known in the art. For example, U.S. Pat. Nos. 3,615,289, 3,645,705, 6,111,055, 6,129,771 and 6,214,063 describe the formulation of candles and pigmented objects embedded in candles referred in the art as “icons.” U.S. Pat. Nos. 3,148,125 and 5,538,718 describe the formulation of lipstick and other cosmetic sticks. U.S. Pat. Nos. 4,275,054, 4,937,069, 5,069,897, 5,102,656 and 5,500,209 each describe the formulation of deodorant and/or antiperspirant. Each of these U.S. Patents is hereby incorporated fully herein by reference.

The amid ester of the invention may be incorporated into commercial products such as those listed above by blending the amid ester with the other components of the product. Typically, the amid ester will be present at a concentration of about 1% to about 50% of the composition, based on the total weight of the composition. It is a routine matter to optimize the amount of the amid ester to have present in a composition, and indeed the amount will vary depending on the actual product and the desired consistency of the product. In general, as more of the amid ester is used in a formulation, the product will display a more pronounced gel character.

In one aspect, the present invention provides a composition, in particular a physiologically acceptable composition, comprising at least one liquid fatty phase structured with the amid ester, combined with at least one amphiphilic compound with a hydrophilic/lipophilic balance (HLB) value of, in various embodiments of the invention, less than 16, less than 14, less than 12, less than 10, less than 9, less than 8, less than 7, less than 6. This composition can be in the form of, e.g., a stick of lipstick. Wax is optionally present in the composition: in one aspect wax is present, while in another aspect wax is absent. The composition demonstrates good impact strength and, when applied to a surface, provides a glossy, non-migrating deposit.

As another example, the present invention provides a mascara product, an eyeliner product, a foundation product, a lipstick product, a deodorant product, a make-up product for the body, a make-up-removing product, an eyeshadow product, a face powder product, a concealer product, a treating shampoo product, a hair conditioning product, an antisun product or a care product for the face or the body comprising: (a) at least one liquid fatty phase comprising: (i) an amid ester and (ii) at least one amphiphilic compound which is liquid at room temperature and which has an HLB value of less than 16, preferably less than 8.

As another example, the present invention provides a structured composition comprising a cosmetically acceptable medium and further comprising: (a) at least one liquid fatty phase comprising at least one structuring polymer which comprises an amid ester; and (b) at least one amphiphilic compound which is liquid at room temperature, with an HLB value of less than 16, preferably less than 8. Optionally, the composition is in cast form, and also optionally the composition is in the form of a mascara product, an eyeliner product, a foundation product, a lipstick product, a deodorant product, a make-up product for the body, a make-up-removing product, an eyeshadow product, a face powder product, a concealer product, a treating shampoo product, a hair conditioning product, an antisun product or a care product for the face or the body.

One or more of the following features may optionally be used to characterize the composition and/or components thereof: the composition may further comprise at least one dyestuff, where optionally: i) the at least one dyestuff is chosen from lipophilic dyes, hydrophilic dyes, pigments and nacres, or ii) the at least one dyestuff is present in a concentration ranging from 0.01% to 40% by weight relative to the total weight of said composition, or the at least one dyestuff is present in a concentration ranging from 5% to 25% by weight relative to the total weight of said composition.

The present invention also provides a cosmetic process for caring for, making up or treating a keratin material comprising the application to at least one keratinous material of a cosmetic composition comprising: (a) at least one liquid fatty phase comprising: (i) at least one amid ester; and (ii) at least one amphiphilic compound which is liquid at room temperature and which has an HLB value of less than 16, preferably less than 8.

The present invention also provides a process of making a cosmetic composition in the form of a physiologically acceptable composition which is glossy and/or non-migrating comprising including in said composition at least one liquid fatty phase, said at least one liquid fatty phase being structured with at least one amid ester and (ii) at least one amphiphilic compound having an HLB value of less than 16, preferably less than 8; wherein said glossy and/or non-migrating cosmetic composition is obtained.

### EXAMPLES

### Raw Material Examples

### Example 1

Monoethanolamine is an item of commerce conforming to the following structure;
H₂N(H)—CH₂CH₂—OH.

### Example 2

Stearic Acid is an item of commerce and conforms to the following structure:
CH₃—(CH₂)₁₆C(O)OH

### Example 3

Behenic Acid is an item of commerce conforming to the following structure:
CH₃—(CH₂)₂₀C(O)OH

### Example 4-7

### Guerbet Acids

Guerbet acids are items of commerce available from Sassol. They conform to the following structure: 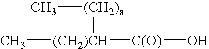
a is an integer ranging from 9 to 17

b is an integer ranging from 7 to 17 with the proviso that b=a+2;

| Example | a | b | Total Carbons in Acid |
|---|---|---|---|
| 4 | 7 | 9 | 20 |
| 5 | 9 | 11 | 24 |
| 6 | 5 | 7 | 16 |
| 7 | 15 | 17 | 36 |

Iso-Acids

Iso-acid are items of commerce available from a variety of suppliers including Cognis.

### Example 8

### Iso-Stearic Acid

Isostearic acid is an item of commerce conforming to the following structure;

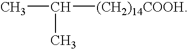

### Example 9

### Iso-Palmitic Acid

Iso-palmitic acid is an item of commerce conforming to the following structure;

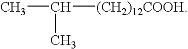

### Hydrocarbons Example 10-16

The hydrocarbons useful in the practice of the present invention are items of commerce available from a variety of sources including Presperse and ExxonMobil.

| Example | Name | CAS Number |
|---|---|---|
| 10 | Isohexadecane | 4390-04-9 |
| 11 | Isoeicosane | 93685-79-1 |
| 12 | Polyisobutene | 9003-29-6 |
| 13 | Isooctane | 540-84-1 |
| 14 | Isododecane | 13475-82-6 |
| 15 | Polyisobutene | 9003-29-6 |
| 16 | Hexane | |
| 17 | Mineral oil | |
| 18 | Dodecane | |

Preparation of the Amid Ester of the Present Invention

General Procedure

To 407.0 grams of monoethanolamine (Example 1) is added the specified number of grams of the specified acid (Examples 2-9). The reaction mass is then heater to between 190° C. and 200° C. and water begins to distill off. The reaction is held at this temperature until the acid value becomes vanishingly small. Nitrogen is sparged through the reaction mass to aid in removal of water and to keep the color light. Vacuum is also slowly applied after about 8 hours at reaction temperature to aid in removal of water.

| Example | Acid | Grams |
|---|---|---|
| 19 | 2 | 5,640.0 |
| 20 | 3 | 6,760.0 |
| 21 | 4 | 6,260.0 |
| 22 | 5 | 7,380.0 |
| 23 | 6 | 5,140.0 |
| 24 | 7 | 10,740.0 |
| 25 | 8 | 5,680.0 |
| 26 | 9 | 5,120.0 |

The products so prepared are waxy materials that lack crystalline structure. They are used without additional purification.

### Examples 27

### Compositions of the Present Invention

To prepare a gel of the invention, the specified number of grams of the specified amid ester is combined with the specified number of grams of the specified hydrocarbon. The two ingredients are taken to elevated temperature, from about 80-150° C., until the amid ester is completely dissolves in the liquid. Upon cooling, the mixture forms the gel of the invention.

| | |
|---|---|
| | Hydrocarbon |
| Amid Ester | Solvent |

| Example | Example | Grams | Example | Grams |
|---|---|---|---|---|
| 23 | 19 | 95.0 | 8 | 5.0 |
| 24 | 20 | 90.0 | 9 | 10.0 |
| 25 | 21 | 85.0 | 10 | 15.0 |
| 26 | 22 | 80.0 | 11 | 20.0 |
| 27 | 23 | 75.0 | 12 | 25.0 |
| 28 | 24 | 70.0 | 13 | 30.0 |
| 29 | 25 | 65.0 | 14 | 35.0 |
| 30 | 26 | 60.0 | 15 | 40.0 |
| 31 | 19 | 50.0 | 16 | 50.0 |

The products of example are clear to slightly hazy gels. They can be used in lipsticks and mascaras by adding pigment, in antiperspirants by adding actives. The gels are cosmetically acceptable on the skin.

All publications and patent applications mentioned in this specification are herein incorporated by reference to the same extent as if each individual publication or patent application was specifically and individually incorporated by reference.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. An amid ester conforming to the following structure;
R—C(O)—N(H)—CH₂CH₂—OC(O)—R
wherein;
R is selected from the group consisting of
(a) alkyl having 18 to 22 carbon atoms;
(b) iso acids;
(c) Guerbet acids 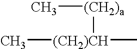
a is an integer ranging from 9 to 17
b is an integer ranging from 7 to 17 with the proviso that b=a+2.

2. An amid ester of claim 1 wherein a is 7 and b is 9.

3. An amid ester of claim 1 wherein a is 9 and b is 11.

4. An amid ester of claim 1 wherein a is 5 and b is 7.

5. An amid ester of claim 1 wherein a is 15 and b is 17.

6. An amid ester of claim 1 wherein R is derived from iso-stearic acid.

7. An amid ester of claim 1 wherein R is derived from iso-palmitic acid.

8. An amid ester of claim 1 wherein R is derived from stearic acid.

9. An amid ester of claim 1 wherein R is derived from behenic acid.

10. A gelled composition comprising;
(i) an amid ester conforming to the following structure;
R—C(O)—N(H)—CH₂CH₂—OC(O)—R
wherein;
R is selected from the group consisting of
(a) alkyl having 18 to 22 carbon atoms;
(b) iso acids;
(c) Guerbet acids 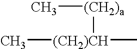
a is an integer ranging from 9 to 17
b is an integer ranging from 7 to 17 with the proviso that b=a+2;
(ii) a hydrocarbon selected from the group consisting of isohexadecane, isoeicosane, polyisobutene, isooctane, isododecane, hexane, decane, dodecane.

11. A gelled composition of claim 10 wherein a is 7 and b is 9.

12. A gelled composition of claim 10 wherein a is 9 and b is 11.

13. A gelled composition of claim 10 wherein a is 5 and b is 7.

14. A gelled composition of claim 10 wherein a is 15 and b is 17.

15. A gelled composition of claim 10 wherein R is derived from iso-stearic acid.

16. A gelled composition of claim 10 wherein R is derived from iso-palmitic acid.

17. A gelled composition of claim 10 wherein R is derived from stearic acid.

18. A gelled composition of claim 10 wherein R is derived from behenic acid.

19. A process for the gellation of composition comprising
(i) an amid ester conforming to the following structure;
R—C(O)—N(H)—CH₂CH₂—OC(O)—R
wherein;
R is selected from the group consisting of
(a) alkyl having 18 to 22 carbon atoms;
(b) iso acids;
(c) Guerbet acids 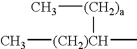
a is an integer ranging from 9 to 17
b is an integer ranging from 7 to 17 with the proviso that b=a+2.
(ii) a hydrocarbon selected from the group consisting of isohexadecane, isoeicosane, polyisobutene, isooctane, isododecane, hexane, decane, dodecane which comprises addition of between 1 and 35% by weight of a branched diamid.
